(19) United States
(12) Patent Application Publication
(10) Pub. No.: US 2010/0186146 A1
(43) Pub. Date: Jul. 29, 2010

(54) EAR COVER WITH DRAWSTRING AND CONCENTRIC RESILIENT AURAL HELIX HANGER

(76) Inventor: Francenia Askew, Plymouth, MA (US)

Correspondence Address: MARK YOUNG, P.A., 12086 FORT CAROLINE ROAD, UNIT 202, JACKSONVILLE, FL, 32225, US

(21) Appl. No.: 12/361,801

(22) Filed: Jan. 29, 2009

(51) Int. Cl. A42B 1/06; A61F 11/00

(52) U.S. Cl. 2/209; 128/866

(57) ABSTRACT

A soft flexible insulating ear cover is provided in the form of a pouch. The cover defines an open compartment for receiving an ear. The pouch has opposite superior and inferior sides. The pouch comprises a soft absorbent medial side and an insulating lateral side opposite the medial side. The medial side further includes an opening adapted to receive an ear. A seam defines a passage around the opening. A resilient tubular helix hanger (also referred to herein as an ear hook) is disposed in the superior side of the passage. A drawstring extends through the passage and through the tubular ear hook and is adapted to controllably close the opening. The interior lining is fusible fleece. The medial side fabric is terry cloth. The lateral side fabric is velvet. The dense pile of velvet provides superior thermal and audible insulation. A layer of quick dry foam batting is preferably provided between the fusible fleece and terry cloth, as well as between the fusible fleece and velvet. The resulting product is washable and reusable.

### FIELD OF THE INVENTION

This invention generally relates to ear coverings, and more particularly, to an ear cover for protecting ears from heat and burns during hairstyling procedures.

### BACKGROUND

Many hairstyling procedures, such as curling, drying and straightening, expose ears to extreme heat. In the case of curling and straightening, extremely hot implements, such as curling irons, hot curlers, and straightening combs, heat the hair to achieve a desired effect. As hair close to ears is styled using these hot implements, accidental contact between the implement and ears is inevitable. The resulting burn is extremely painful, unsightly, may lead to infection and take several days to heal.

Likewise, hair dryers create burning risks and also pose a nuisance. Specifically, as is well known, hair dryers subject ears to intense heat, especially when hot air is directed near the ears for any extended period of time. Burning caused by a hair dryer is gradual and may be difficult to detect until it is too late. Burning and drying of the middle ear may severely compromise hearing. Concomitantly, hair dryers expose ears to high noise levels. The noise is not only a nuisance, but may trigger a severe headache and contribute to noise induced hearing loss.

Over the years, to shield ears from burns, various ear protectors have been conceived. A common thread among the various conceptions is a covering for the ears. A key differentiating feature among the various coverings is the mechanism to secure the coverings to the ears, e.g., an elastic cord surrounding the opening, as in U.S. Pat. No. 346,175; an elastic band for engaging a portion of the ear, as in U.S. Pat. No. 1,826,309; a curved periphery to engage the helix of the ear, as in U.S. Pat. No. 1,845,689; an elastic band with a flared skirt around the opening, as in U.S. Pat. No. 6,944,886; an envelope-style covering, as in U.S. Pat. No. 6,912,733; an elastic-cord and drawstring, as in U.S. Pat. No. 6,298,493; a c-shaped closable ribbon, as in U.S. Pat. No. 5,898,945; and an elastic band, as in U.S. Pat. No. 5,778,455. While the various coverings are generally effective for their intended purposes, i.e., protecting ears, they do not reliably and comfortably maintain engagement with the ear. This is due in part due to misalignment and lack of sufficient structural support and binding over key parts of the ear. Many of the closures are uncomfortable and tend to loosen as a wearer moves his or her head.

What is needed is a comfortable, easy to use, ear covering device that securely engages the ear. The invention is directed to overcoming one or more of the problems and solving one or more of the needs as set forth above.

### SUMMARY OF THE INVENTION

To solve one or more of the problems set forth above, in one aspect of an exemplary implementation of the invention, a soft flexible insulating cover is provided in the form of a pouch. The cover defines an open compartment for receiving an ear. The pouch has opposite superior and inferior sides. The pouch comprises a soft absorbent medial side and an insulating lateral side opposite the medial side. The medial side further includes an opening adapted to receive an ear. A seam defines a passage around the opening. A tubular helix hanger (also referred to herein as an ear hook) is disposed in the superior side of the passage. A drawstring extends through the passage and through the tubular ear hook and is adapted to controllably close the opening. The tubular helix hanger comprises a resilient elastomeric arcuate tube, semicircular in shape, with an outer diameter of ¼ to ½ inch and an inner diameter no less than the diameter of the drawstring, and a radius of curvature of 1 to 3 inches.

In another aspect of the invention, the drawstring, i.e., an elongate string with a pair of ends, is provided to cinch the opening around an ear. The drawstring extends through a sheath-like circumferential passage defined in the cover around the periphery of the opening. The free ends of the drawstring pass through openings or eyelets in the cover and through a retaining clip adapted to clamp engaged portions of the free ends. The drawstring may be tightened by pulling on the free ends and bunching up the encircled material to cinch an engaged ear by reducing the size of the opening.

In another aspect of the invention, the tubular helix hanger is provided in the sheath-like circumferential passage defined in the cover around the periphery of the opening. The tubular helix hanger is preferably molded from 5/16-inch OD tubular elastomeric material, semicircular in-shape with a radius of curvature of about two (2) inches. The tubular helix hanger has a hollow channel extending from one opening to an opposite opening. The drawstring is threaded through the hollow channel of the tubular helix hanger, with one free end of the drawstring extending from one opening and the other free end of the drawstring extending from the opposite opening. The tubular helix hanger may be comprised of an elastomer such as natural rubber, polyisoprene, butyl rubber, halogenated butyl rubbers, polybutadiene, styrene-butadiene rubber, nitrile rubber, hydrated nitrile rubber, chloroprene rubber, ethylene propylene rubber, ethylene propylene diene rubber, epichlorohydrin rubber, polyacrylic rubber, silicone rubber, fluorosilicone rubber, fluoroelastomers, perfluoroelastomers, tetrafluoro ethylene/propylene rubber, chlorosulfonated polyethylene, ethylene-vinyl acetate, and/or

In another aspect of the invention, the heat-resistant elastomeric tubular arcuate tubular helix hanger is positioned in the superior portion of the sheath-like circumferential passage opposite the openings/eyelets. The tubular helix hanger may be affixed in position in the passage, such as with fabric glue, to prevent the tubular helix hanger from drifting along the passage during use.

In another aspect of the invention, the interior lining is fusible fleece, a soft napped insulating synthetic wool fabric made from synthetic fibers, such as polyethylene terephthalate (PET). The medial side fabric is terry cloth, fabric with loops that can absorb large amounts of liquids. The lateral side fabric is velvet, tufted fabric in which the cut threads are very evenly distributed, with a short dense pile made from silk, cotton or synthetics such as polyester, viscose, acetate and mixtures of different synthetics, or synthetics and natural fibers. The dense pile of velvet provides superior thermal and audible insulation. A layer of quick dry foam batting is preferably provided between the fusible fleece and terry cloth, as well as between the fusible fleece and velvet. The resulting product is washable and reusable.

### BRIEF DESCRIPTION OF THE DRAWINGS

The foregoing and other aspects, objects, features and advantages of the invention will become better understood with reference to the following description, appended claims, and accompanying drawings, where:

FIG. 1 provides a medial side view of an ear cover with a drawstring and concentric resilient aural helix hanger according to principles of the invention; and

FIG. 2 provides a lateral side view of an ear cover with a drawstring and concentric resilient aural helix hanger according to principles of the invention; and

FIG. 3 provides a lateral side view of an ear cover with a drawstring and concentric resilient aural helix hanger worn by a person according to principles of the invention; and

FIG. 4 provides a medial side view of an ear cover with a drawstring and concentric resilient aural helix hanger worn on an ear according to principles of the invention; and

FIG. 5A provides a medial superior portion view of an ear cover with a drawstring and concentric resilient aural helix hanger according to principles of the invention; and

FIG. 5B provides a cross sectional view of an ear cover with a drawstring and concentric resilient aural helix hanger according to principles of the invention; and

FIG. 6 provides a perspective view of a heat-resistant elastomeric tubular arcuate ear hook according to principles of the invention.

Those skilled in the art will appreciate that the figures are not intended to be drawn to any particular scale; nor are the figures intended to illustrate every embodiment of the invention. The invention is not limited to the exemplary embodiments depicted in the figures or the shapes, relative sizes, ornamental aspects or proportions shown in the figures.

As used herein, the terms medial, lateral, superior and inferior are used in an anatomical sense. The term superior means toward the head or the upper part of a structure, while inferior refer to the lower part of a structure or away from the head. Medial means toward the midline of the body and lateral away from that midline.

### DETAILED DESCRIPTION

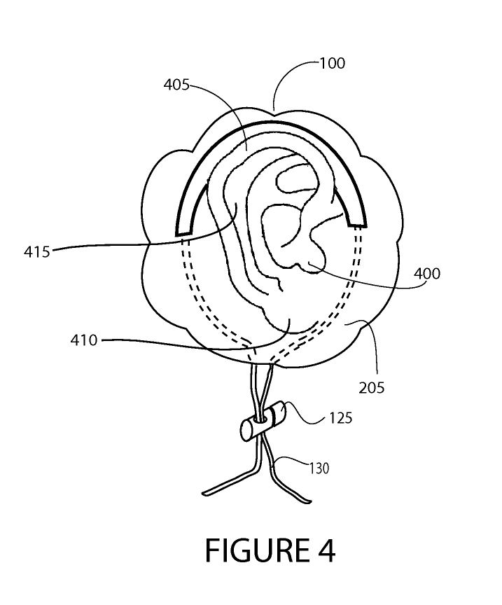

Referring to the Figures, in which like parts are indicated with the same reference numerals, various views of an exemplary an ear cover with a drawstring and concentric resilient aural helix hanger according to principles of the invention are shown. Referring first to FIG. 4, an outer ear 400 is conceptually shown. The outer ear 400 includes a pinna 415 (which is Latin for feather), the flesh covered cartilage appendage on either side of the head. The pinna 415 is also commonly referred to as the auricle or auricular. The prominent rim of the pinna 415 is called the helix 405. The earlobe 410 (lobulus auricult, also known as lobe or lobule) is the soft lowermost portion of the human pinna. The earlobe 410 is composed of tough areolar and adipose (fatty) connective tissues, lacking the firmness and elasticity of the rest of the pinna 415 including the helix 405, since the earlobe 410 contains no cartilage.

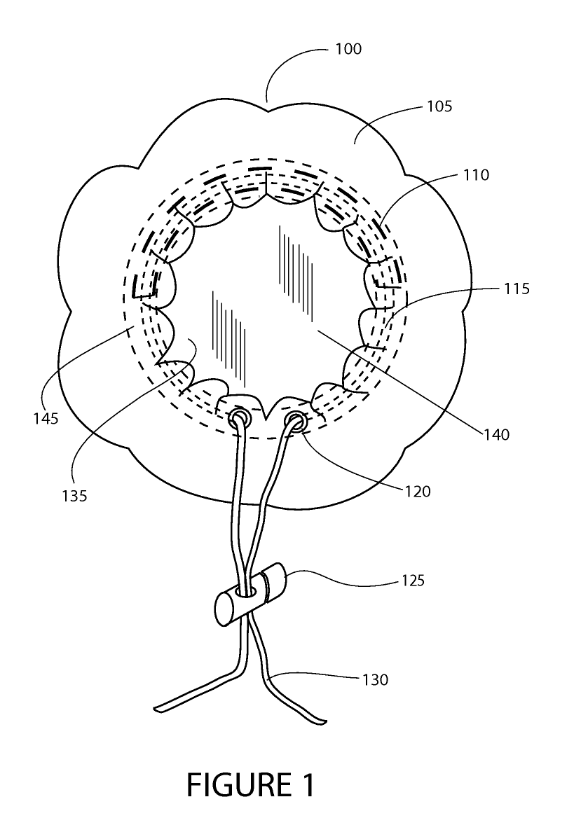

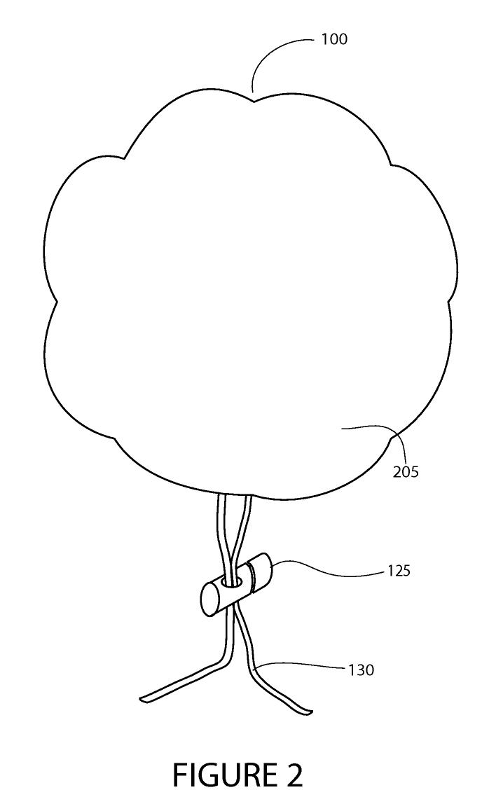

Referring now to FIGS. 1 and 2, medial and lateral side view of an exemplary ear cover 100 with a drawstring 130 and concentric resilient aural helix hanger 110 according to principles of the invention is shown. The exemplary ear cover 100 comprises a soft, flexible, insulating cover in the form of a pouch 105 (i.e., a container), with an open compartment 135 accessed through a medial side opening 140 for receiving an ear 400 (i.e., an exterior or outer ear), a soft absorbent medial side 105 and an insulating lateral side 205.

A drawstring 130, i.e., an elongate string with a pair of ends, is provided to cinch the opening 140 around an ear. The drawstring 130 extends through a seam defining a sheath-like circumferential passage 145 in the cover 105 around the periphery of the opening 140. The free ends of the drawstring 130 pass through openings or eyelets 120 in the cover 100 and through a retaining clip 125 adapted to clamp engaged portions of the free ends. The drawstring 130 may be tightened by pulling on the free ends and bunching up the encircled material to cinch an engaged ear by reducing the size of the opening 140. Thus, the drawstring 130 controllably reduces and expands the size of the opening 140 to engage or release an ear.

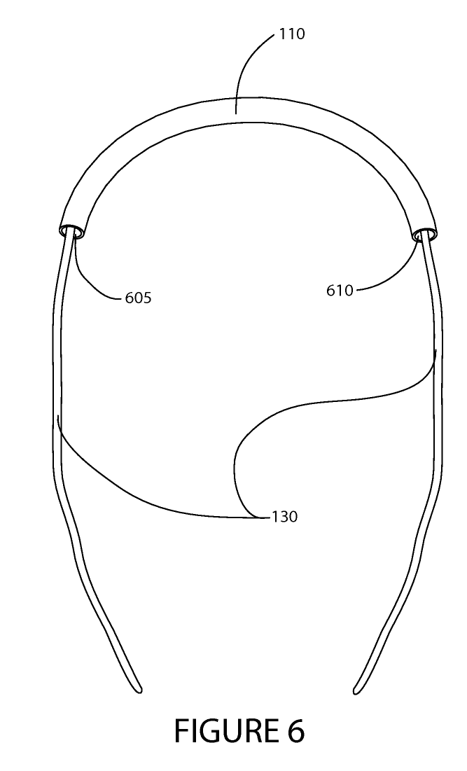

A heat-resistant elastomeric tubular arcuate ear hook 110 is provided in the sheath-like circumferential passage 145 defined in the cover 100 around the periphery of the opening 140. The ear hook 110 is preferably molded from ¼-inch to ½-inch outer diameter (OD), more preferably 5/16-inch OD, tubular elastomeric material, semicircular in shape with a radius of curvature of about 1 to 3 inches, more preferably 2 inches. The inner diameter is equal to or greater than the diameter of the drawstring (i.e., the maximum cross-sectional profile dimension of the drawstring), which is less than the OD. The ear hook 110 has a hollow channel extending from one opening 605 to an opposite opening 610. As shown in FIGS. 1 and 6, the drawstring 130 is threaded through the hollow channel of the ear hook 110, with one free end of the drawstring 130 extending from one opening 605 and the other free end of the drawstring 130 extending from the opposite opening 610. In an exemplary embodiment, the ear hook 110 is comprised of an elastomer such as natural rubber, polyisoprene, butyl rubber, halogenated butyl rubbers, polybutadiene, styrene-butadiene rubber, nitrile rubber, hydrated nitrile rubber, chloroprene rubber, ethylene propylene rubber, ethylene propylene diene rubber, epichlorohydrin rubber, polyacrylic rubber, silicone rubber, fluorosilicone rubber, fluoroelastomers, perfluoroelastomers, tetrafluoro ethylene/propylene rubber, chlorosulfonated polyethylene, ethylene-vinyl acetate, and/or

The heat-resistant elastomeric tubular arcuate ear hook 110 is positioned in the superior portion of the sheath-like circumferential passage 145 opposite the openings/eyelets 120, as shown in FIG. 1. In a preferred embodiment, the ear hook 110 is affixed in position in the passage 145, such as with fabric glue, to prevent the ear hook 110 from drifting along the passage 145 during use.

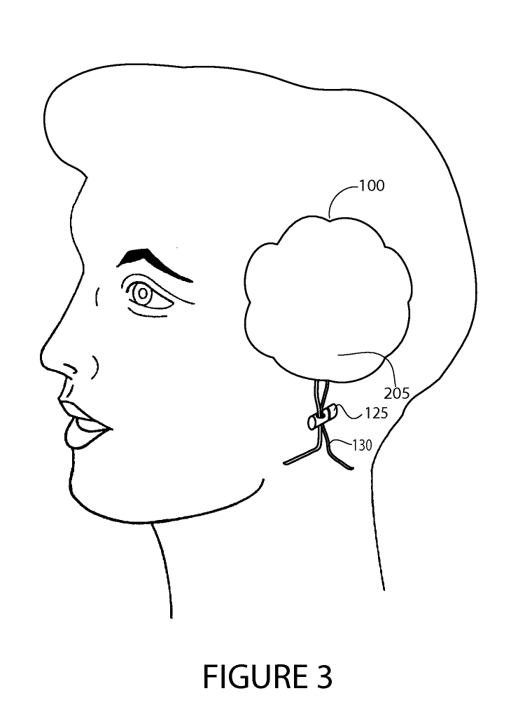

With reference to FIGS. 3 and 4, during use, the cover 100 is placed over an ear with the ear hook 110 rests on the cartilage of the pinna 415 behind the superior helix 405 of the pinna 415, as shown in FIG. 4. Tightening the drawstring 130 causes the ear hook 110 to bend and conform to the engaged portion of the ear, without binding. Concomitantly, tightening the drawstring 130 causes the periphery of the inferior portion of the opening 610 to engage the cartilage of the pinna 415 behind the earlobe. When the drawstring 130 is loosened, the ear hook 110 relaxes and returns to its undeformed shape.

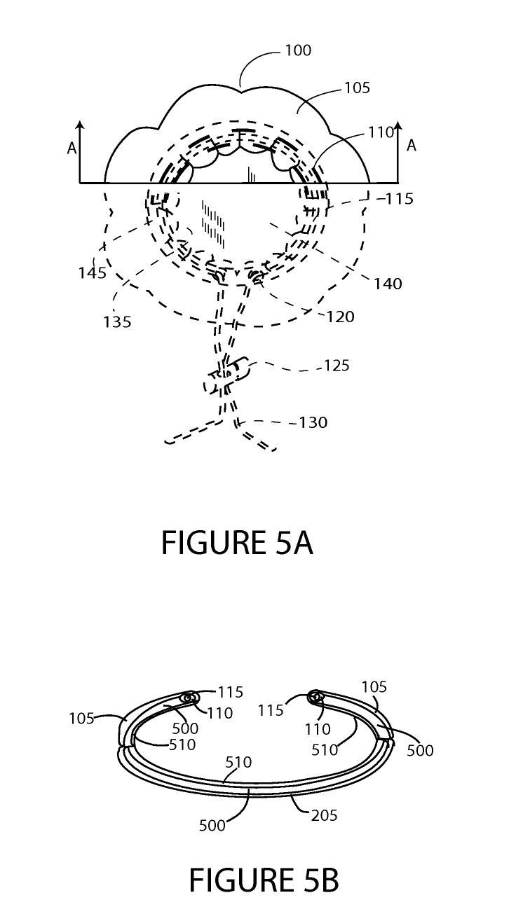

An important aspect of the exemplary embodiment is the unique composition of the cover 100. The materials provide superior comfort and insulation without interfering with operability. With reference to the medial superior portion view of FIG. 5A and the cross sectional view of FIG. 5B, the interior lining is preferably fusible fleece 510, a soft napped insulating synthetic wool fabric made from synthetic fibers, such as polyethylene terephthalate (PET). The medial side fabric is preferably terry cloth 105, fabric with loops that can absorb large amounts of liquids. The lateral side fabric may be velvet 205, tufted fabric in which the cut threads are very evenly distributed, with a short dense pile made from silk, cotton or synthetics such as polyester, viscose, acetate and mixtures of different synthetics, or synthetics and natural fibers. The dense pile of velvet 205 provides superior thermal and audible insulation. A layer of quick dry foam batting 500 is preferably provided between the fusible fleece and terry cloth, as well as between the fusible fleece and velvet. The resulting product is washable and reusable.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A soft flexible insulating cover in the form of a pouch, said cover defining an open compartment configured for receiving an ear, said pouch having opposite superior and inferior sides, said pouch comprising a soft absorbent medial side and an insulating lateral side opposite said medial side, the medial side further including an opening adapted to receive an ear, a seam defining a passage around the opening, a tubular helix hanger disposed in the superior side of said passage, and a drawstring extending through said passage and through said tubular helix hanger and adapted for controllably closing the opening.

2. A soft flexible insulating cover according to claim 1, wherein the tubular helix hanger comprises an elastomeric arcuate tube.

3. A soft flexible insulating cover according to claim 1, wherein the tubular helix hanger comprises an elastomeric arcuate tube, semicircular in shape.

4. A soft flexible insulating cover according to claim 1, said drawstring having a diameter, wherein the tubular helix hanger comprises an elastomeric arcuate tube semicircular in shape and having an outer diameter of ¼ to ½ inch and an inner diameter no less than the diameter of the drawstring.

5. A soft flexible insulating cover according to claim 1, said drawstring having a diameter, wherein the tubular helix hanger comprises an elastomeric arcuate tube semicircular in shape and having an outer diameter of ¼ to ½ inch, and an inner diameter no less than the diameter of the drawstring, and a radius of curvature of 1 to 3 inches.

6. A soft flexible insulating cover according to claim 1, said drawstring having a diameter, wherein the tubular helix hanger comprises an elastomeric arcuate tube semicircular in shape and having an outer diameter of 5/16 of an inch, and an inner diameter no less than the diameter of the drawstring, and a radius of curvature of 2 inches.

7. A soft flexible insulating cover according to claim 1, wherein the tubular helix hanger comprises an elastomeric arcuate tube affixed to the channel.

8. A soft flexible insulating cover according to claim 1, said drawstring having a pair of free ends, and said passage having a pair of passage openings, each of said passage openings including an eyelet.

9. A soft flexible insulating cover according to claim 1, wherein the tubular helix hanger comprises an elastomeric arcuate tube comprising an elastomer from the group consisting of natural rubber, polyisoprene, butyl rubber, halogenated butyl rubbers, polybutadiene, styrene-butadiene rubber, nitrile rubber, hydrated nitrile rubber, chloroprene rubber, ethylene propylene rubber, ethylene propylene diene rubber, epichlorohydrin rubber, polyacrylic rubber, silicone rubber, fluorosilicone rubber, fluoroelastomers, perfluoroelastomers, tetrafluoro ethylene/propylene rubber, chlorosulfonated polyethylene and ethylene-vinyl acetate.

10. A soft flexible insulating cover according to claim 1, said drawstring having a pair of free ends, and said free ends of said drawstring passing through a retaining clip.

11. A soft flexible insulating container defining an open compartment configured for receiving an ear, said container having opposite superior and inferior sides, said container comprising a soft absorbent medial side and an insulating lateral side opposite said medial side, and an interior liner, said medial side further including an opening adapted to receive an ear, a seam defining a passage around the opening, a resilient tubular helix hanger disposed in the superior side of said passage, and a drawstring extending through said passage and through said tubular helix hanger and adapted for controllably closing the opening, said soft absorbent material comprising terry cloth.

12. A soft flexible insulating container according to claim 11, wherein the resilient tubular helix hanger comprises an elastomeric arcuate tube, and said insulating lateral side comprising velvet.

13. A soft flexible insulating container according to claim 11, wherein the resilient tubular helix hanger comprises an elastomeric arcuate tube, semicircular in shape, and said interior liner comprises fleece.

14. A soft flexible insulating container according to claim 11, said drawstring having a diameter, wherein the resilient tubular helix hanger comprises an elastomeric arcuate tube semicircular in shape and having an outer diameter of ¼ to ½ inch and an inner diameter no less than the diameter of the drawstring, and said interior liner comprises fusible fleece.

15. A soft flexible insulating container according to claim 11, said drawstring having a diameter, wherein the resilient tubular helix hanger comprises an elastomeric arcuate tube semicircular in shape and having an outer diameter of ¼ to ½ inch, and an inner diameter no less than the diameter of the drawstring, and a radius of curvature of 1 to 3 inches.

16. A soft flexible insulating container according to claim 11, said drawstring having a diameter, wherein the resilient tubular helix hanger comprises an elastomeric arcuate tube semicircular in shape and having an outer diameter of 5/16 of an inch, and an inner diameter no less than the diameter of the drawstring, and a radius of curvature of 2 inches.

17. A soft flexible insulating container according to claim 11, wherein the resilient tubular helix hanger comprises an elastomeric arcuate tube affixed to the channel.

18. A soft flexible insulating container according to claim 11, said drawstring having a pair of free ends, and said passage having a pair of passage openings, each of said passage openings including an eyelet.

19. A soft flexible insulating container according to claim 11, wherein the resilient tubular helix hanger comprises an elastomeric arcuate tube comprising an elastomer from the group consisting of natural rubber, polyisoprene, butyl rubber, halogenated butyl rubbers, polybutadiene, styrene-butadiene rubber, nitrile rubber, hydrated nitrile rubber, chloroprene rubber, ethylene propylene rubber, ethylene propylene diene rubber, epichlorohydrin rubber, polyacrylic rubber, silicone rubber, fluorosilicone rubber, fluoroelastomers, perfluoroelastomers, tetrafluoro ethylene/propylene rubber, chlorosulfonated polyethylene and ethylene-vinyl acetate.

20. A soft flexible insulating container according to claim 11, said drawstring having a pair of free ends, and said free ends of said drawstring passing through a retaining clip.
